(19) United States
(12) Patent Application Publication
(10) Pub. No.: US 2024/0158724 A1
(43) Pub. Date: May 16, 2024

(54) Jet Fuel Wash

(71) Applicant: Kevin Charles Miller, Chattanooga, TN (US)

(72) Inventor: Kevin Charles Miller, Chattanooga, TN (US)

(21) Appl. No.: 18/388,719

(22) Filed: Nov. 10, 2023

Related U.S. Application Data
(63) Continuation-in-part of application No. 17/553,015, filed on Dec. 16, 2021, now abandoned.

(51) Int. Cl. C11D 3/50; C11D 1/29; C11D 3/382; C11D 17/04

(52) U.S. Cl. CPC C11D 3/50; C11D 1/29; C11D 3/382; C11D 17/049

(57) ABSTRACT

Disclosed herein are liquid cleansing compositions comprising: a. from about 90 percent by volume to about 99 percent by volume of a base liquid soap; b. from about 0.2 percent by volume to about 1.5 percent by volume Citronella oil; c. from about 0.05 percent by volume to about 1.0 percent by volume Peppermint oil; d. from about 0.1 percent by volume to about 1.0 percent by volume Eucalyptus oil; e. from about 0.1 percent by volume to about 1.0 percent by volume Sandalwood oil; f. from about 0.1 percent by volume to about 1.0 percent by volume Lemon oil; and g. from about 0.25 percent by volume to 2.5 percent by volume of an abrasive material, wherein the combined volume percent value of all components does not exceed 100 wt. %, and all volume percent values are based on the total volume of the composition.

### CROSS-REFERENCE TO RELATED APPLICATION

This application is a continuation-in-part of U.S. patent application Ser. No. 17/553,015 filed Dec. 16, 2021, which is incorporated herein in its entirety by this reference thereto.

### FIELD OF THE DISCLOSURE

The present disclosure relates generally to a cleaning product. More specifically, the present disclosure pertains to a cleaning formulation for use at least on skin and cloth materials.

### BACKGROUND

Jet fuel was engineered to have a powerful odor and to not dissipate easily. The odor takes days to dissipate from a person's skin, and even longer for one's hair. When it gets on one's clothes, one must leave them outside for typically more than a week. It may take more than a week for the smell to dissipate from clothing and washing with other clothes may cause the scent to be absorbed by those clothes. Additionally, it may require one's clothes to be left outside until the smell has dissipated. Conventional cleaning products do little to resolve the problem.

Additionally, prolonged exposure of jet fuel to skin can be harmful. Effective removal of the substance is needed as a safety precaution for people that are exposed to the substance on a regular basis. No product exists to solve this problem.

Thus, there is a need for a cleaning product that is safe to use on skin but strong enough to remove any trace of pungently aromatic hydrocarbons.

### SUMMARY

The above-described and other deficiencies of the art are met by liquid cleansing compositions comprising: from about 90 percent by volume to about 99 percent by volume of a base liquid soap; from about 0.2 percent by volume to about 1.5 percent by volume Citronella oil; from about 0.05 percent by volume to about 1.0 percent by volume Peppermint oil; from about 0.1 percent by volume to about 1.0 percent by volume Eucalyptus oil; from about 0.1 percent by volume to about 1.0 percent by volume Sandalwood oil; from about 0.1 percent by volume to about 1.0 percent by volume Lemon oil; and from about 0.25 percent by volume to 2.5 percent by volume of an abrasive material, wherein the combined volume percent value of all components does not exceed 100 wt. %, and all volume percent values are based on the total volume of the composition.

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present disclosure is defined by the claims.

The above described and other features are exemplified by the following detailed description, examples, and claims.

The present disclosure is directed towards specific (liquid) compositions or formulations for a soap or cleanser that eliminates the extremely stubborn smell of jet fuel. Some aspects of the present disclosure may comprise a base liquid soap made for scale production mix with natural essential oils that are not commonly used in soap that are combined in the right percentages such that they are completely skin safe.

### BRIEF DESCRIPTION OF FIGURES

The present disclosure is described in detail below with reference to the attached drawing figures, wherein:

FIG. 1 illustrates a flowchart providing a method in accordance with aspects of the present disclosure.

### DETAILED DESCRIPTION OF ILLUSTRATIVE ASPECTS

The present disclosure may be understood more readily by reference to the following detailed description of desired aspects and the examples included therein. In the following specification and the claims that follow, reference will be made to a number of terms which have the following meanings.

Various combinations of elements of this disclosure are encompassed by this disclosure, for example, combinations of elements from dependent claims that depend upon the same independent claim.

Some aspects of the present disclosure are described with specificity herein to meet statutory requirements. However, the scope of the disclosure is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

The present disclosure is directed towards soap compositions that eliminates the strong smell of jet fuel. Some aspects of the present disclosure may comprise a base liquid soap made for scale production combined with specific amounts of natural essential oils that are not commonly used in soap. The soap composition is completely skin safe. Safety for the skin may be determined by a number of known standards or methods, including, but not to be limited to skin patch tests and skin irritation tests.

Some aspects of the present disclosure may be produced by combining a base liquid soap and an abrasive material with at least one of the following essential oils: Citronella oil, Peppermint oil, Eucalyptus oil, Sandalwood oil, and Lemon oil to produce a liquid cleansing composition. In some aspects, the liquid cleansing composition may comprise from about 90 percent by volume to about 99.5 percent volume, or from about 95.5 percent by volume to about 99.5 percent volume of a base liquid soap, 96 percent by volume to about 99.5 percent volume, or 97 percent by volume to about 99.5 percent volume of a base liquid soap. In some aspects, the liquid cleansing composition may comprise from about 0.2 to 1.5 percent by volume, or from about 0.2 to 1.3, about 0.3 to 1.4, about 0.5 to 1.0, about 0.2 to 0.4, or about 0.2 to 0.45 percent by volume of Citronella essential oil. In some aspects, the liquid cleansing composition may comprise from about 0.05 to 1.0, about 0.05 to 0.1, about 0.05 to 0.5, about 0.05 to 0.8, about 0.05 to 0.5, about 0.08 to 0.8, about 0.08 to 0.1, about 0.06 to 0.1, or from about 0.05 to 0.3 percent by volume of a peppermint essential oil. In further aspects, the liquid cleansing composition may comprise from about 0.1 to 1.0, about 0.1 to 0.8, about 0.1 to 0.7, about 0.15 to 0.2, about or about 0.15 to 0.22 percent by volume of a eucalyptus essential oil. In yet further aspects, the liquid cleansing composition may comprise from about 0.1 to 1.0, about 0.1 to 0.8, about 0.1 to 0.7, about 0.15 to 0.2, about 0.15 to 0.22 percent by volume sandalwood essential oil. In yet further aspects, the liquid cleansing composition may comprise from about 0.1 to 1.0, about 0.1 to 0.8, about 0.1 to 0.7, about 0.15 to 0.2, or about 0.15 to 0.22 of lemon essential oil. In still further aspects, the liquid cleansing composition may comprise about 0.25 to 2.5, about 0.2 to 2, about 0.2 to 1.5, about 0.2 to 1.8, about 0.2 to 1.0, about 0.2 to 0.8, about 0.3 to 0.5, about 0.3 to 0.8, about 0.3 to 1.0 percent by volume of abrasive material.

Specifically, the liquid cleansing composition may comprise from about 95 percent by volume to about 99.5 percent by volume of a base soap combined with Citronella oil in a range of 0.2 to 1.5 percent by volume, Peppermint oil in a range of 0.05 to 1.0 percent by volume, Eucalyptus oil in a range of 0.1 to 1.0 percent by volume, Sandalwood oil in a range of 0.1 to 1.0 percent by volume, Lemon oil in a range of 0.1 to 1.0 percent by volume, and the abrasive material in a range of 0.25 to 2.5 percent by volume. More specifically, the liquid cleansing composition may comprise a base soap combined with Citronella oil in a range of 0.2 to 0.3 percent by volume, Peppermint oil in a range of 0.05 to 0.1 percent by volume, Eucalyptus oil in a range of 0.1 to 0.2 percent by volume, Sandalwood oil in a range of 0.1 to 0.2 percent by volume, Lemon oil in a range of 0.1 to 0.2 percent by volume, and an abrasive material in a range of 0.25 to 0.50 percent by volume. At least one acceptable combination of the aforementioned ingredients has been determined to be the base liquid soap combined with 0.23 percent by volume Citronella oil, 0.06 percent by volume Peppermint oil, 0.11 percent by volume Eucalyptus oil, 0.11 percent by volume Sandalwood oil, 0.11 percent by volume Lemon oil, and 0.4 percent by volume abrasive material. The skilled artisan will realize that the balance of the cleansing composition is a percent volume of the base liquid soap to provide a cleansing composition that is 100 percent by volume.

The base liquid soap may refer to a liquid suspension soap base or a general purpose soap. In some examples, the base liquid soap may be propylene glycol based or sulfate based or a combination thereof. A suitable base soap is commercially available as Crafter's Choice™. The abrasive material may include, but not be limited to, pumice, pulverize or powdered nut shells, such as almond shells or walnut shells, other cellulose-based exfoliants and the like or combinations thereof. One having skill in the art will recognize that concentrations of more than 5.5 percent by volume essential oils (i.e., Citronella oil, Peppermint oil, Eucalyptus oil, Sandalwood oil, Lemon oil) may not be safe for continuous use directly on skin. As such, formulations of the present disclosure for direct use on skin include no more than 6 percent by volume total essential oil, no more than 5.8 percent by volume total essential oils, no more than 5.5 percent by volume total essential oils, no more than 5.2 percent by volume total essential oils, or no more than 3 percent by volume total essential oils.

Some aspects of the present disclosure may include shower soap, shampoo, laundry detergent and the like. Some aspects of the present disclosure may include handwipes which are highly portable and can be kept near a mechanic's workspace for use immediately after exposure to jet fuel or other hydrocarbon liquid. Such a handwipe may comprise a liquid absorbent and disposable hand tower that may be saturated with the cleaning product described herein. Such a disposable hand tower may comprise paper, cotton, woven bamboo fiber, or the like or combinations thereof.

The disclosed liquid cleansing composition may be used to remove jet fuel from hard and porous or biological surfaces, such as skin, clothing, or textiles. The disclosed composition may exhibit desirable performance for removing and/or reducing jet fuel on surfaces based on odor analysis and an extractable petroleum hydrocarbons (EPH) with flame ionization detection (FID) method and suitable characterization. As an example, the disclosed composition may achieve at least 20%, at least 30%, at least 40%, at least 50%, at least 60%, at least 70%, at least 80%, at least 85%, at least 88%, at least 90%, at least 95%, at least 99% improvement in removing and/or reducing jet fuel on a surface. As a yet further example, the disclosed composition may achieve at least 20%, at least 30%, at least 40%, at least 50%, at least 60%, at least 70%, at least 80%, at least 85%, at least 88%, at least 90%, at least 95%, at least 99% improvement in removing and/or reducing jet fuel when used for handwashing performed in compliance with applicable Occupational Safety Health Administration (OSHA) or World Health Organization (WHO) handwashing standards compared to handwashing with water alone or handwashing with a conventional soap. As a still further example, the disclosed composition may achieve at least 80%, at least 85%, at least 88%, at least 90%, at least 95%, at least 99% improvement in removing and/or reducing jet fuel when used for handwashing performed in compliance with applicable OSHA or WHO handwashing standards compared to handwashing with a conventional soap. As a still further example, the disclosed composition may achieve 20%, at least 30%, at least 40%, at least 50%, at least 60%, at least 70%, at least 80%, at least 85%, at least 88%, at least 90%, at least 95%, at least 99% improvement in removing and/or reducing jet fuel when used for handwashing performed in compliance with applicable OSHA or WHO handwashing standards compared to handwashing with water alone. Improvement may be characterized by measuring the amount of jet fuel, or the amount of distinctive jet fuel components (namely, certain hydrocarbons), in fluid collected from handwashing with the disclosed composition compared to handwashing with water alone or handwashing with a conventional soap. Measurement of the amount of jet fuel, or distinctive jet fuel hydrocarbons, may be obtained via EPH methods with flame ionization detection (FID) characterization.

In further aspects, the disclosed liquid cleansing composition may eliminate the extremely stubborn smell of jet fuel from a surface, such as solid surfaces or porous or biological surfaces. These liquid cleansing compositions may be more effective in the removal or washing away of jet fuel from hard surfaces or porous surfaces than water alone or than conventional soaps when used under the same conditions. In specific examples, the liquid cleansing composition may result in less jet fuel present on the cleansed hands from handwashing with the disclosed formulation for at least 10 seconds, 15 seconds, or at least 20 seconds. Less jet fuel present on the cleansed hands may be evidenced or characterized by greater jet fuel content in fluid collected from handwashing with the disclosed liquid cleansing composition. Jet fuel content in the fluid collected from handwashing may be evaluated according to a number of suitable methods. In a specific example, an extractable petroleum hydrocarbon (EPH) in liquid with flame ionization detection (FID) may be used to measure the characteristic hydrocarbon content of jet fuel apparent in the fluid collected from handwashing.

In yet further examples, handwashing with the disclosed composition may result in greater jet fuel present in fluid collected from the handwashing compared to fluid collected from handwashing with water alone or with a conventional soap. Fluid retained from handwashing with the disclosed formulation exhibits greater amounts of jet fuel (characterized as hydrocarbons) when compared to the fluid retained from handwashing with tap water or when compared to fluid retained from handwashing with an alternative or conventional soap. Direct observance of skin washed with the disclosed formulation after treatment with a volume of jet fuel may exhibit less odor than skin rinsed or washed with water alone or a conventional soap after treatment with a volume of jet fuel.

Handwashing standards may be in compliance with Occupational Safety and Health Administration (OSHA) standards. Proper OSHA hand hygiene procedures include wetting hands with water; applying sufficient soap to cover all surfaces; thoroughly washing all parts of hands and fingers up to the wrists, rubbing hands together for at least 20 seconds, and rinsing hands with water. Toney-Butler TJ, Gasner A, Carver N. Hand Hygiene. July 31, 2023. In: StatPearls [Internet]. Treasure Island (FL): StatPearls Publishing; January 2023. Available from: https://www.ncbi.nlm.nih.gov/books/NBK470254/ WHO Guidelines on Hand Hygiene in Health Care: First Global Patient Safety Challenge Clean Care Is Safer Care. World Health Organization; Geneva: 2009.

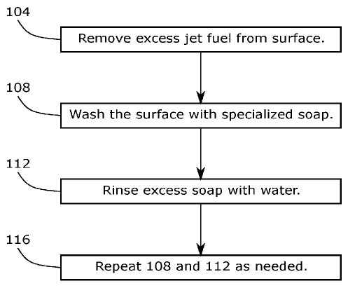

FIG. 1 illustrates a flowchart 100 providing an exemplary method in accordance with aspects of the present disclosure. At step 104, excess jet fuel and/or other hydrocarbon liquid, if any, is removed from the surface. As used herein, “surface” is a broad term that includes, but is not limited to floors, countertops, parts, tools, clothing, hair, hands, skin, or the like. At step 108, washing the surface affected by the hydrocarbon liquid with a specialize soap comprising base liquid soap, citronella oil, peppermint oil, eucalyptus oil, sandalwood oil, lemon oil, and abrasive material. At step 112, the excess soap may be rinsed from the surface with water. At step 116, steps 108 through 112 may be repeated as needed. Initially, the surface may be moistened with water prior to applying the specialized soap.

Aspects of the present disclosure have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

### Definitions

It is also to be understood that the terminology used herein is for the purpose of describing particular aspects only and is not intended to be limiting. As used in the specification and in the claims, the term “comprising” can include the aspects “consisting of” and “consisting essentially of” Unless defined otherwise, all technical and scientific terms used herein have the same meaning as commonly understood by one of ordinary skill in the art to which this disclosure belongs. In this specification and in the claims, which follow, reference will be made to a number of terms which shall be defined herein.

As used in the specification and the appended claims, the singular forms “a,” “an” and “the” include plural referents unless the context clearly dictates otherwise. Thus, for example, reference to “an essential oil” includes mixtures of two or more essential oil components. As used herein, the term “combination” is inclusive of blends, mixtures, f, reaction products, and the like.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optional additional processes” means that the additional processes can or cannot be included and that the description includes methods that both include and that do not include the additional processes.

As used herein, “surface” is a broad term that includes, but is not limited to floors, countertops, parts, tools, clothing, hair, hands, skin, or the like.

As used herein, “essential oil” may refer to plants essential oils which are a volatile extracts from the flower, leaf, stem, root or fruit of a plant. Generally, essential oils (or, as also referred herein as the given plant “oil”) is a secondary metabolite of plants consisting of simple compounds of lower molecular weights and having certain aromatic characteristics.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding aspects to parts by weight of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

References in the specification and concluding aspects to volume percent of a particular element or component in a composition or article, denotes the volume relationship between the element or component and any other elements or components in the composition or article for which a volume percent is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A volume percent of a component, unless specifically stated to the contrary, is based on the total volume of the formulation or composition in which the component is included.

As used herein the terms “weight percent,” “wt %,” and “wt. %,” which can be used interchangeably, indicate the percent by weight of a given component based on the total weight of the composition, unless otherwise specified. That is, unless otherwise specified, all wt % values are based on the total weight of the composition. It should be understood that the sum of wt % values for all components in a disclosed composition or formulation is 100.

As used herein the terms “volume percent,” “vol %,” and “vol. %,” which can be used interchangeably, indicate the percent by volume of a given component based on the total weight of the composition, unless otherwise specified. That is, unless otherwise specified, all vol% values are based on the total weight of the composition. It should be understood that the sum of vol % values for all components in a disclosed composition or formulation is 100.

Unless otherwise stated to the contrary herein, all test standards are the most recent standard in effect at the time of filing this application.

Each of the materials disclosed herein are either commercially available and/or the methods for the production thereof are known to those of skill in the art. It is understood that the compositions disclosed herein have certain functions.

### Aspects of the Disclosure

In various aspects, the present disclosure pertains to and includes at least the following aspects.

Aspect 1. A liquid cleansing composition for removing a hydrocarbon liquid or film from a surface, comprising: from about 90 percent by volume to about 99 percent by volume of a base liquid soap; from about 0.2 percent by volume to about 1.5 percent by volume Citronella oil; from about 0.05 percent by volume to about 1.0 percent by volume Peppermint oil; from about 0.1 percent by volume to about 1.0 percent by volume Eucalyptus oil; from about 0.1 percent by volume to about 1.0 percent by volume Sandalwood oil; from about 0.1 percent by volume to about 1.0 percent by volume Lemon oil; and from about 0.25 percent by volume to 2.5 percent by volume of an abrasive material, wherein the combined volume percent value of all components does not exceed 100 wt. %, and all volume percent values are based on the total volume of the composition.

Aspect 2. The liquid cleansing composition of claim 1, wherein the percent by volume of Citronella oil ranges from 0.2 to 0.3 percent, the percent by volume of Peppermint oil ranges from 0.05 to 0.1 percent, the percent by volume of Eucalyptus oil ranges from 0.1 to 0.2 percent, the percent by volume of Sandalwood oil ranges from 0.1 to 0.2 percent, the percent by volume of Lemon oil ranges from 0.1 to 0.2 percent, and the percent by volume of the abrasive material ranges from 0.25 to 0.5 percent.

Aspect 3. The liquid cleansing composition of claim 1, wherein the percent by volume of Citronella oil is 0.23 percent, the percent by volume of Peppermint oil ranges from 0.05 to 0.1 percent, the percent by volume of Eucalyptus oil ranges from 0.1 to 0.2 percent, the percent by volume of Sandalwood oil ranges from 0.1 to 0.2 percent, the percent by volume of Lemon oil ranges from 0.1 to 0.2 percent, and the percent by volume of the abrasive material ranges from 0.25 to 0.5 percent.

Aspect 4. The liquid cleansing composition of claim 1, wherein the percent by volume of Citronella oil is 0.23 percent, the percent by volume of Peppermint oil ranges is 0.06 percent, the percent by volume of Eucalyptus oil is 0.11 percent, the percent by volume of Sandalwood oil is 0.11 percent, the percent by volume of Lemon oil is 0.11 percent, and the percent by volume of the abrasive material is 0.4 percent.

Aspect 5. The liquid cleansing composition of claim 1, wherein the abrasive material is pumice, pulverized nut shells or powdered nut shells.

Aspect 6. The liquid cleansing composition of claim 5, wherein the nut shells are almond shells or walnut shells.

Aspect 7. The liquid cleansing composition of claim 1, wherein handwashing with composition removes at least 20%, 30%, 40%, 50%, 60%, 70%, 80%, 85%, 90%, 95%, or 99% more jet fuel or jet fuel characteristic components when compared to handwashing with water alone or handwashing with a conventional soap, wherein the handwashing is conducted according to OSHA or WHO handwashing standards and an amount of removal is determined by measuring jet fuel content in accordance with EPH FID methods in fluid collected after the handwashing.

Aspect 8. The liquid cleansing composition of claim 1, wherein handwashing with the composition removes at least 80%, 85%, 90%, 95%, or 99% more jet fuel or jet fuel characteristic components when compared to handwashing with water alone, wherein handwashing is conducted according to OSHA or WHO handwashing standards and an amount of removal is determined by measuring jet fuel content in accordance with EPH FID methods in fluid collected after the handwashing.

Aspect 9. The liquid cleansing composition of claim 1 wherein handwashing with the composition removes at least 20%, 30%, 40%, 50%, 60%, 70%, 80%, 85%, 90%, 95%, or 99% more jet fuel or jet fuel characteristic components when compared to handwashing with a conventional soap, wherein handwashing is conducted according to OSHA or WHO handwashing standards and an amount of removal is determined by measuring jet fuel content in accordance with EPH FID methods in fluid collected after the handwashing.

Aspect 7. A hand towel comprising the liquid cleansing composition of claim 1, wherein the hand towel is liquid absorbent and disposable.

Aspect 8. A method of removing a hydrocarbon liquid or film from a surface, comprising: removing excess hydrocarbon liquid or film from the surface; applying a liquid cleansing composition to the surface, wherein the liquid cleansing composition comprises; from about 90 percent by volume to about 99 percent by volume of a base liquid soap; from about 0.2 percent by volume to about 1.5 percent by volume Citronella oil; from about 0.05 percent by volume to about 1.0 percent by volume Peppermint oil; from about 0.1 percent by volume to about 1.0 percent by volume Eucalyptus oil; from about 0.1 percent by volume to about 1.0 percent by volume Sandalwood oil; from about 0.1 percent by volume to about 1.0 percent by volume Lemon oil; and from about 0.25 percent by volume to 2.5 percent by volume about of an abrasive material, rinsing the liquid cleansing composition from the surface with water; repeating steps b and c if necessary to remove any residual hydrocarbon liquid or film.

Aspect 9. The method of claim 8, comprising pretreating the surface with water after removing excess hydrocarbon liquid or film and before applying the liquid cleansing composition.

Aspect 10. The method of claim 8, wherein the percent by volume of Citronella oil is 0.23 percent, the percent by volume of Peppermint oil ranges from 0.05 to 0.1 percent, the percent by volume of Eucalyptus oil ranges from 0.1 to 0.2 percent, the percent by volume of Sandalwood oil ranges from 0.1 to 0.2 percent, the percent by volume of Lemon oil ranges from 0.1 to 0.2 percent, and the percent by volume of the abrasive material ranges from 0.25 to 0.5 percent.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (for example, amounts, temperature, etc.), but some errors and deviations should be accounted for. Unless indicated otherwise, parts are parts by volume, temperature is in ° C. or is at ambient temperature, and pressure is at or near atmospheric. Unless indicated otherwise, percentages referring to composition are in terms of vol %.

There are numerous variations and combinations of mixing conditions, for example, component concentrations, temperatures, and other mixing ranges and conditions that can be used. Only reasonable and routine experimentation will be required to optimize such process conditions.

The composition described below may be formed and tested. It is expected that this composition would exhibit desirable performance for removing and/or reducing jet fuel on surfaces based on odor analysis and an extractable petroleum hydrocarbons (EPH) with flame ionization detection (FID) method and suitable characterization. This composition includes the components presented in Table 1. The disclosed components may be combined, for example, by mixing them at room temperature.

TABLE 1
Prophetic Example.

| Component | Amount | |
|---|---|---|
| Base liquid soap (propylene glycol based | 15 | ounces |
| sulfate soap) | | |
| Citronella oil | 1 | ml |
| Peppermint oil | 0.25 | ml |
| Eucalyptus oil | 0.5 | ml |
| Sandalwood oil | 0.5 | ml |
| Lemon oil | 0.5 | ml |
| Abrasive material (walnut shell powder) | 50 | grams |

For testing, a twenty (20) milliliter volume of the formulation is applied to individual user's hands classified as Analyst-1 and Analyst-2. Analyst-1 hands are rinsed in tap water for a duration of 15 to 30 seconds and liquid retained after rinsing. Analyst-2 hands are washed with a two (2) gram amount of the liquid cleansing composition formulation for 15 to 30 seconds and the liquid retained after washing. Analyst-3 hands are washed under the same or similar conditions with a conventional hand soap. Comparative testing is performed with handwashing with an equal amount of an alternative or conventional soap for 15 to 30 seconds. Handwashing is compliant with OSHA standards. Prophetic results are summarized below.

Odor analysis. Fluid used for handwashing is collected and assessed for odor per FCC Direct Observance. With each handwashing session using the disclosed formulation as compared to tap water, the characteristic odor of jet fuel on the washed hands is less apparent.

TABLE 2
Observed odor.

| | After Wash 1 | After Wash 2 | After Wash 4 |
|---|---|---|---|
| Analyst-1 | Odor very strong | Odor very strong | Odor very strong |
| Analyst-2 | Odor less apparent | Odor less apparent | No apparent odor |
| Analyst-3 | Odor strong | Odor strong | Odor strong |

EPH FID. Fluid used for handwashing is collected and extracted with a suitable solvent. The fluid retained from handwashing with the disclosed formulation exhibits higher amounts of jet fuel (characterized as hydrocarbons) when compared to the fluid retained from handwashing with tap water or when compared to fluid retained from handwashing with an alternative or conventional soap. Handwashing with the disclosed formulation removes or washes away at least 20%, 30%, 40%, 50%, 60%, 70%, 80% more, 85%, 90%, 95%, 98%, 99%, 99.2%, 99.5% more jet fuel (or jet fuel characteristic components) than handwashing with water alone or with a conventional soap.

Aspects of the present disclosure have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

The above description is intended to be illustrative, and not restrictive. For example, the above-described examples (or one or more aspects thereof) may be used in combination with each other. Other aspects can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed aspect. Thus, the following claims are hereby incorporated into the Detailed Description as examples or aspects, with each claim standing on its own as a separate aspect, and it is contemplated that such aspects can be combined with each other in various combinations or permutations. The scope of the disclosure should be determined with reference to the appended claims, along with the full scope of equivalents to which such claims are entitled.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims. It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

What is claimed is:

1. A liquid cleansing composition for removing a hydrocarbon liquid or film from a surface, comprising:
a. from about 90 percent by volume to about 99 percent by volume of a base liquid soap;
b. from about 0.2 percent by volume to about 1.5 percent by volume Citronella oil;
c. from about 0.05 percent by volume to about 1.0 percent by volume Peppermint oil;
d. from about 0.1 percent by volume to about 1.0 percent by volume Eucalyptus oil;
e. from about 0.1 percent by volume to about 1.0 percent by volume Sandalwood oil;
f. from about 0.1 percent by volume to about 1.0 percent by volume Lemon oil; and
g. from about 0.25 percent by volume to 2.5 percent by volume of an abrasive material,
wherein the combined volume percent value of all components does not exceed 100 wt. %, and all volume percent values are based on the total volume of the composition.

2. The liquid cleansing composition of claim 1, wherein the percent by volume of Citronella oil ranges from 0.2 to 0.3 percent, the percent by volume of Peppermint oil ranges from 0.05 to 0.1 percent, the percent by volume of Eucalyptus oil ranges from 0.1 to 0.2 percent, the percent by volume of Sandalwood oil ranges from 0.1 to 0.2 percent, the percent by volume of Lemon oil ranges from 0.1 to 0.2 percent, and the percent by volume of the abrasive material ranges from 0.25 to 0.5 percent.

3. The liquid cleansing composition of claim 1, wherein the percent by volume of Citronella oil is 0.23 percent, the percent by volume of Peppermint oil ranges from 0.05 to 0.1 percent, the percent by volume of Eucalyptus oil ranges from 0.1 to 0.2 percent, the percent by volume of Sandalwood oil ranges from 0.1 to 0.2 percent, the percent by volume of Lemon oil ranges from 0.1 to 0.2 percent, and the percent by volume of the abrasive material ranges from 0.25 to 0.5 percent.

4. The liquid cleansing composition of claim 1, wherein the percent by volume of Citronella oil is 0.23 percent, the percent by volume of Peppermint oil ranges is 0.06 percent, the percent by volume of Eucalyptus oil is 0.11 percent, the percent by volume of Sandalwood oil is 0.11 percent, the percent by volume of Lemon oil is 0.11 percent, and the percent by volume of the abrasive material is 0.4 percent.

5. The liquid cleansing composition of claim 1, wherein the abrasive material is pumice, pulverized nut shells or powdered nut shells.

6. The liquid cleansing composition of claim 5, wherein the nut shells are almond shells or walnut shells.

7. A hand towel comprising the liquid cleansing composition of claim 1, wherein the hand towel is liquid absorbent and disposable.

8. A method of removing a hydrocarbon liquid or film from a surface, comprising:
a. removing excess hydrocarbon liquid or film from the surface;
b. applying a liquid cleansing composition to the surface, wherein the liquid cleansing composition comprises;
i. from about 90 percent by volume to about 99 percent by volume of a base liquid soap;
ii. from about 0.2 percent by volume to about 1.5 percent by volume Citronella oil;
iii. from about 0.05 percent by volume to about 1.0 percent by volume Peppermint oil;
iv. from about 0.1 percent by volume to about 1.0 percent by volume Eucalyptus oil;
v. from about 0.1 percent by volume to about 1.0 percent by volume Sandalwood oil;
vi. from about 0.1 percent by volume to about 1.0 percent by volume Lemon oil; and
vii. from about 0.25 percent by volume to 2.5 percent by volume about of an abrasive material,
c. rinsing the liquid cleansing composition from the surface with water;
d. repeating steps b and c if necessary to remove any residual hydrocarbon liquid or film.

9. The method of claim 8, comprising pretreating the surface with water after removing excess hydrocarbon liquid or film and before applying the liquid cleansing composition.

10. The method of claim 8, wherein the percent by volume of Citronella oil is 0.23 percent, the percent by volume of Peppermint oil ranges from 0.05 to 0.1 percent, the percent by volume of Eucalyptus oil ranges from 0.1 to 0.2 percent, the percent by volume of Sandalwood oil ranges from 0.1 to 0.2 percent, the percent by volume of Lemon oil ranges from 0.1 to 0.2 percent, and the percent by volume of the abrasive material ranges from 0.25 to 0.5 percent.
